(19) United States
(12) Patent Application Publication
(10) Pub. No.: US 2014/0257389 A1
(43) Pub. Date: Sep. 11, 2014

(54) PERCUTANEOUS BREAK OFF ROD

(71) Applicant: BLACKSTONE MEDICAL, INC., Lewisville, TX (US)

(72) Inventors: John Lovell, North Bergen, NJ (US); Stephen Termyna, Boonton, NJ (US)

(73) Assignee: BLACKSTONE MEDICAL, INC., Lewisville, TX (US)

(21) Appl. No.: 13/793,114

(22) Filed: Mar. 11, 2013

(51) Int. Cl. A61B 17/70

(52) U.S. Cl. CPC A61B 17/705; A61B 17/7002; A61B 17/7083; A61B 17/7049
USPC 606/246; 606/86 A

(57) ABSTRACT

A break off rod for percutaneous insertion with a spinal assembly, such that the break off rod includes a rod body. The break off rod also includes a connector, in which the connector is configurable to be connected with a fastener an insertion device, such that the insertion device abuts against a spinal assembly when the rod body of the break off rod is properly inserted in the spinal assembly. The break off rod also includes a break off region, in which the break off region connects the rod body and the connector. The break off region is configurable to break off the connector from the rod body when a force is applied by the insertion device.

### TECHNICAL FIELD

The present disclosure relates in general to medical device technology, and more specifically to a break off rod for percutaneous insertion with a spinal assembly.

### BACKGROUND

Generally, a rod body is inserted with a spinal assembly with an insertion device that clamps or attaches to an end of the rod body. In some cases, sophisticated insertion devices are required to properly grasp and release the end portion of the rod body. Further, the end of the rod body that was held by the insertion device significantly sticks out and overhangs from the spinal assembly. A rod body that overhangs or sticks out from the posterior spinal assembly may cause irritation of rubbing against the bones or cartilage and it may also cause facet impingement There remains a need for insertion of a rod body with a spinal assembly that does not cause the rod body to significantly overhang or stick out from the spinal assembly.

### SUMMARY

In accordance with the present disclosure, a break off rod is provided for percutaneous insertion with a spinal assembly, such that the break off rod includes a rod body. The break off rod also includes a connector, in which the connector is configurable to be connected with an opening of a face of an insertion device, such that the face of the insertion device abuts against a spinal assembly when the rod body of the break off rod is inserted in the spinal assembly. The break off rod also includes a break off region, in which the break off region connects the rod body and the connector. The break off region is configurable to break off the connector from the rod body when a force is applied by the insertion device.

In accordance with the present disclosure, a insertion device is provided for percutaneous insertion of a break off rod with a spinal assembly. The insertion device includes a body, where a face of the body has an opening to receive a connector of a break off rod for percutaneous insertion with a spinal assembly. The break off rod includes a rod body, a connector, and a break off region, where the face of the insertion body is configurable to lock in the connector of the break off rod within the insertion body. The face of the insertion body abuts against the spinal assembly when the rod body of the break off rod is inserted in the spinal assembly. The insertion body is configurable to break off the connector from the rod body prior to removal of the insertion body.

The present disclosure provides several important technical advantages. In certain embodiments, the present disclosure provides a break off rod that allows for the rod body to be positioned within the spinal assembly, such that the rod body does not overhang or stick out significantly from the spinal assembly. A rod body that overhangs or sticks out significantly from the spinal assembly may cause irritation of rubbing against the bones or cartilage and it may also cause facet impingement Thus, the present disclosure prevents and/or reduces the negative effects caused by a rod body that overhangs or sticks out from the spinal assembly. Further, the present disclosure allows for the percutaneous insertion of the rod at a lower profile, which is less invasive to the patient. Accordingly, a patient's surgery time and recovery time may be reduced.

Other technical advantages of the present disclosure will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the features and advantages of the present disclosure, reference is now made to the detailed description along with the accompanying figures and in which:

FIG. 1A is a perspective view of a percutaneous break off rod according to one embodiment of the present disclosure;

FIG. 1B is another perspective view of the percutaneous break off rod according to one embodiment of the present disclosure;

FIG. 1C is a perspective view of an inserter device for the percutaneous break off rod according to one embodiment of the present disclosure;

FIG. 2A is a perspective view of the insertion of a rod body of a percutaneous break off rod in a posterior spinal assembly according to one embodiment of the present disclosure;

FIG. 2B is a perspective view of the removal of a connector of a percutaneous break off rod in a posterior spinal assembly according to one embodiment of the present disclosure;

FIG. 3A is a perspective view of a percutaneous break off rod according to one embodiment of the present disclosure;

FIG. 3B is another perspective view of the percutaneous break off rod according to one embodiment of the present disclosure; and

FIG. 3C is a perspective view of an inserter device for the percutaneous break off rod according to one embodiment of the present disclosure.

### DETAILED DESCRIPTION

While various embodiments of the break off rod 100 according to the present disclosure are discussed in detail below, it should be appreciated that the present disclosure provides many applicable inventive concepts that can be embodied in a wide variety of specific contexts. Without limiting the scope of the present disclosure, the break off rod 100 is described in connection with percutaneous insertion with an embodiment of a posterior spinal assembly. However, break off rod 100 can be used with other spinal assemblies. In some embodiments, the break off rod 100 described herein may also be used in anterior, anterolateral, or posterolateral procedures. The specific embodiments discussed herein are merely illustrative of specific ways to make and use the break off rod 100 disclosed herein and do not delimit the scope of the application, and their usage does not delimit the application, except as outlined in the claims.

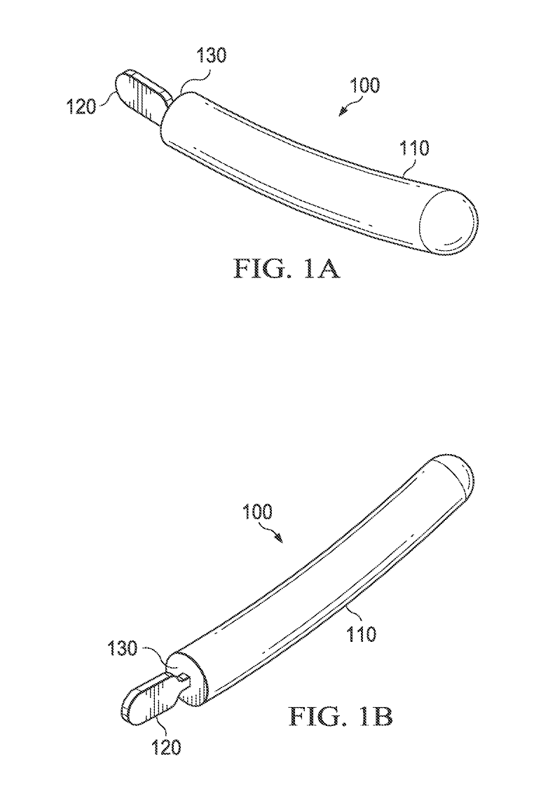

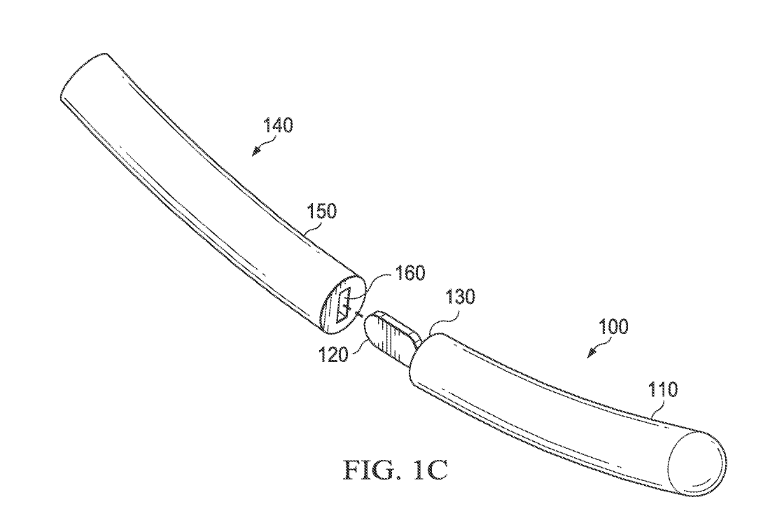

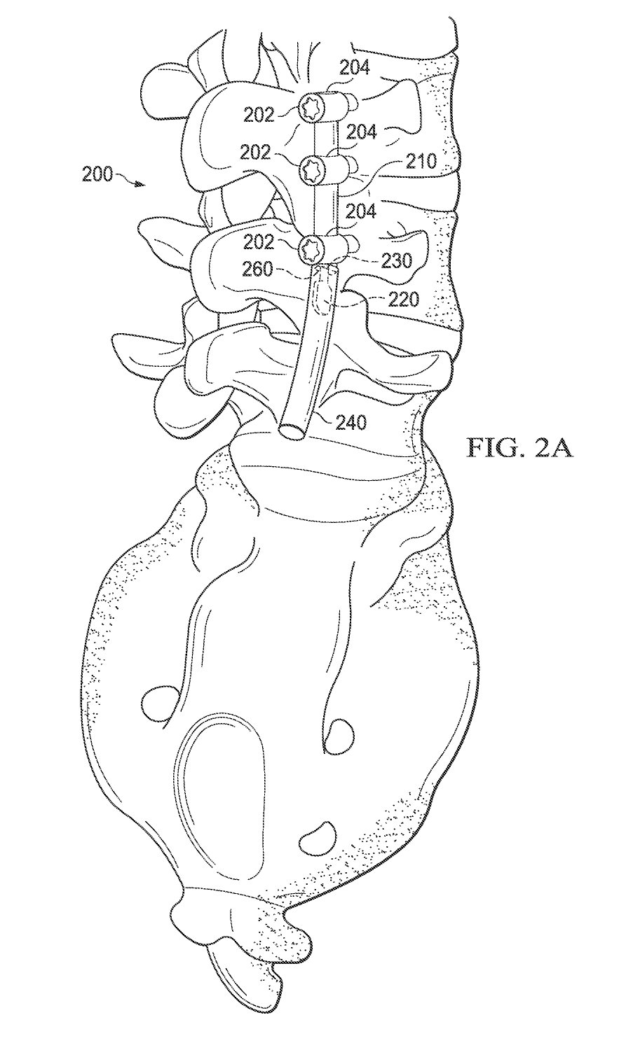

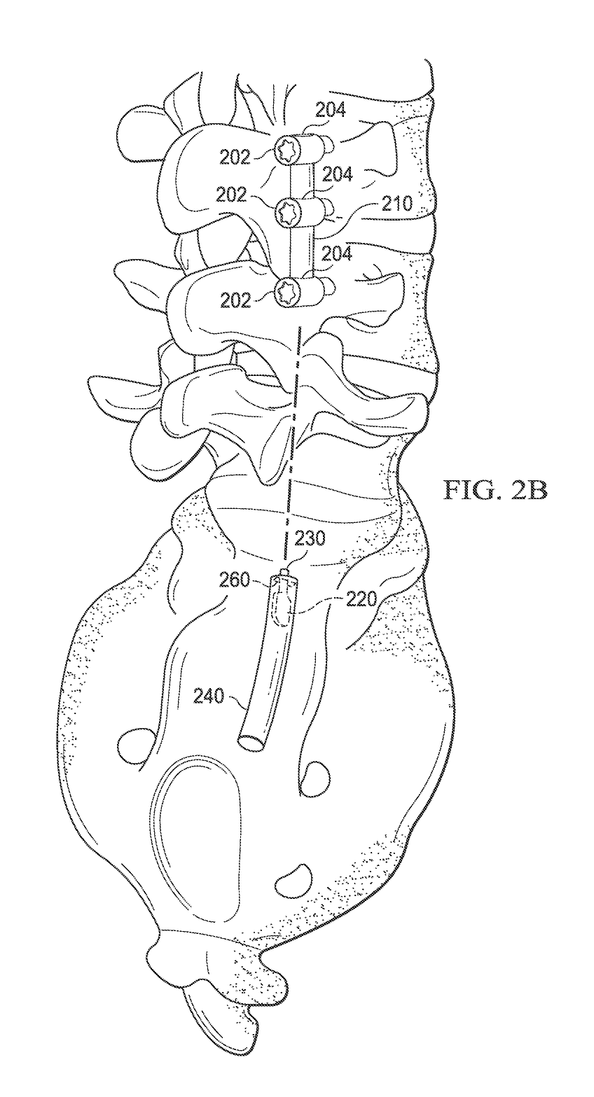

FIG. 1A is a perspective view of a percutaneous break off rod 100 according to one embodiment of the present disclosure. Break off rod 100 may have a rod body 110, a connector, 120, and a break off region 130. Rod body 100 can be percutaneously inserted during a procedure for insertion as the longitudinal member in a spinal assembly screwed into one or more vertebrae, which is explained in more detail below in FIGS. 2A and 2B. Rod body 110 may be any desired shape and length that allows it to be inserted into the desired spinal assembly.

Break off region 130 is the portion of break off rod 100 that is configurable and designed to break when a force is applied to connector 120. Break off region 130 may be made of any desired material or design that allows for connector 120 to break off from rod body 120 when an appropriate force is applied. In some embodiments, a rotational force may cause the break off region 130 to break off connector 120 from rod body 110. In some embodiments, break off region can be designed such that a non-rotational force (e.g., an up and down movement, left to right movement, other movements, other forces, etc.) may cause break off region to break off connector 120 from rod body 110.

FIG. 1B is another perspective view of the percutaneous break off rod 100 having a rod body 110, a connector 120, and a break off region 130 according to one embodiment of the present disclosure.

FIG. 1C is a perspective view of an inserter device 140 for the percutaneous break off rod 100 according to one embodiment of the present disclosure. Break off rod 100 can have a rod body 110, a connector, 120, and a break off region 130. Inserter device 140 can have an inserter body 150 and a fastener 160.

Prior to the percutaneous insertion of break off rod 100, connector 120 is connected to fastener 160. In the illustrated embodiment, connector 120 is a flat wedge shape that may be inserted into fastener 160, which is an opening designed specifically to receive the flat wedge shape of connector 120. In the illustrated example, fastener 160 may be designed in any number of ways, such that connector 120 is fitted or locked into place with fastener 160. For example, fastener 160 may have an opening with dimensions very close to the dimensions of connector, such that the friction between connector 120 and fastener 160 causes the connector 120 and 160 to be locked into place with one another. In another example, fastener 160 may have a hollow interior within inserter body 150 with grooves for receiving connector 120, such that connector 120 may be rotated ninety degrees after being inserted in fastener 160 to fit within the grooves of inserter body 150, such that connector 120 is locked into place with fastener 160.

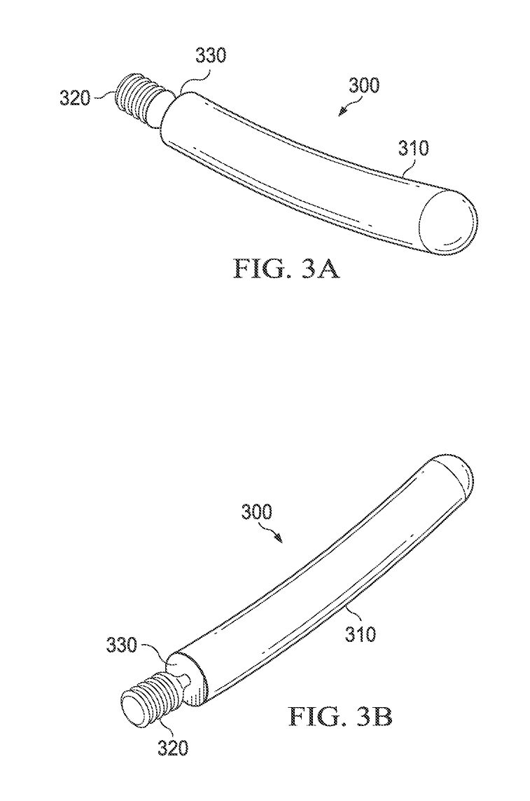

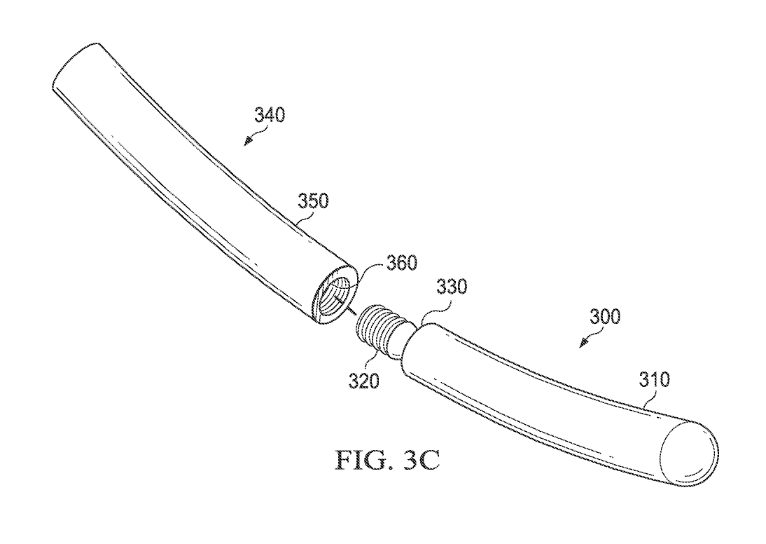

Although the illustrated embodiment of connector 120 includes a flat wedge shape region, any shape or design may be used to carry out the teachings in this disclosure that allow for connector 120 to be connected to fastener 160. For example, connector 120 may be shaped as a round wedge, a star, a ‘V’, a hexagon, etc. Although the illustrated embodiment of connector 120 includes a flat wedge shape region, any shape or design may be used to carry out the teachings in this disclosure that allow for connector 120 to be connected to fastener 160. By having the connector 120 secured and locked to fastener 160, connector 120 can be safely removed by insertion device 140, such that no foreign bodies are left in the patient's body after rod body 110 is broken off. An embodiment of connector 120 having threads that is threaded into fastener 160 having cooperating threads is illustrated below in FIGS. 3A, 3B, and 3C.

Although the illustrated embodiment of fastener 160 includes an opening for connecting to connector 120, any design may be used to carry out the teachings in this disclosure that allow for connector 120 to be connected to fastener 160. For example, fastener 160 may include devices or members on the outside of inserter body 150 rather than an opening to connect to connector 120.

Inserter body 150 may be any desired shape and length that allows for the percutaneous insertion of rod body 150 to be inserted into the desired spinal assembly. In some embodiments, the circumference of inserter body 110 and/or fastener 160 are larger than the circumference of rod body 110, which allows for the inserter body 150 and/or fastener 160 to abut against the spinal assembly that receives the rod body 110 and indicates that rod body 110 is in the correct position. In some embodiments (e.g., embodiments where connector 120 attaches to fastener 160 located on the exterior of inserter body 150, the circumference of connector 120 is larger than the circumference of rod body 110, which allows for the connector 120 to abut against the spinal assembly that receives the rod body 110 and indicates that rod body 110 is in the correct position. In some embodiments, the break off region 130, the connector 120, and/or the inserter body 140 have a larger diameter than the diameter of the aperture of the bone screw opening of the spinal assembly receiving the rod body 110 to ensure that the rod body 110 does not overhang or extend from the spinal assembly. Thus, break off region 130 and connector 120 allow for insertion of rod body 110 in the spinal assembly, such that the rod body 110 does not such that rod body 110 does not overhang or stick out from the spinal assembly. A rod body that overhangs or sticks out from a spinal assembly may cause irritation of rubbing against the bones or cartilage and it may also cause facet impingement Thus, the present disclosure prevents and/or reduces the negative effects caused by a rod body that overhangs or sticks out from the spinal assembly. Further, the present disclosure allows for the percutaneous insertion of the rod at a lower profile because the insertion device 140 can be of a circumference only slightly larger than the circumference of rod body 110, which is less invasive to the patient. Accordingly, a patient's surgery time and recovery time may be reduced.

FIG. 2A is a perspective view of the insertion of the rod body 110 of the percutaneous break off rod in a posterior spinal assembly according to one embodiment of the present disclosure. The illustrated example of a posterior spinal assembly includes three bone screws 202 driven into the pedicles. The bone screws 202 have openings 204 to receive rod body 210. In some embodiments, the screw heads may allow for multi-axial positioning. After rod body 210 is properly positioned, the screw heads may be tightened to lock rod body 210 into place to form the spinal assembly. Although spinal assembly is illustrated as being a posterior spinal assembly having three screws, rod body 110 (and the entire percutaneous break off rod) can be used with any type of spinal assembly having any number of bone screws of any type to carry out the teachings of this disclosure.

FIG. 2B is a perspective view of the removal of the connector 220 of the percutaneous break off rod in a posterior spinal assembly according to one embodiment of the present disclosure.

In operation, connector 220 may be connected to fastener 260 before the procedure. A physician may create an incision in posterior (back) of patient. The incision may only need to be as large as the width of the insertion device 240. The physician inserts the inserter device 240 and the connected break off rod while guiding the rod body 210 through the screw openings 204 until the inserter device 240 abuts against the bone screw of the posterior spinal assembly. Once the inserter device 240 abuts against the screw, the rod body 210 is positioned properly, such that the rod body 210 itself is not overhanging or sticking out from the spinal assembly.

After the rod body 210 is properly positioned, the heads of the bone screws can be used to lock the rod body 210 into place with the spinal assembly. The physician can apply a force (e.g., a rotational force) to the insertion device 240, which causes the break off region 230 to break and separate the rod body 210 from connector 220 (and insertion device 240). After the connector 220 is separate from rod body 210, rod body is positioned properly, such that rod body 210 does not extend or overhang from the spinal assembly. Further, the separated connector 220 remains securely attached to insertion device, such that the separated connector 220 is removed from the patient's body along with the insertion device 240.

FIG. 3A is a perspective view of a percutaneous break off rod 300 according to one embodiment of the present disclosure. In the illustrated embodiment, connector 320 is designed having a threaded cylindrical region.

FIG. 3B is another perspective view of the percutaneous break off rod 300 according to one embodiment of the present disclosure.

FIG. 3C is a perspective view of an inserter device 340 for the percutaneous break off rod 300 according to one embodiment of the present disclosure. In the illustrated embodiment, fastener 360 is designed having a threaded interior region, such that the threaded connector 320 can be screwed into fastener 360.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A break off rod for percutaneous insertion with a spinal assembly, the break off rod comprising:
a rod body;
a connector, wherein the connector is connected to a fastener of an insertion device, wherein the insertion device abuts against a spinal assembly when the rod body of the break off rod is properly inserted in the spinal assembly;
a break off region, wherein the break off region connects the rod body and the connector, and wherein the break off region is configurable to break off the connector from the rod body when a force is applied by the insertion device.

2. The break off rod of claim 1, wherein the connector of the break off rod is cylindrical and threaded and the fastener of the insertion device is an open area having threads to fasten the connector to the insertion device.

3. The break off rod of claim 1, wherein the connector of the break off rod is shaped as a flat wedge and the fastener of the insertion device is shaped to frictionally fasten the flat wedge shaped connector to the insertion device.

4. The break off rod of claim 1, wherein the break off rod breaks off the connector from the rod body when a rotational force is applied to the insertion device.

5. The break off rod of claim 1, wherein the break off rod breaks off the connector from the rod body when a non-rotational force is applied to the insertion device.

6. The break off rod of claim 1, wherein the break off region has a smaller circumference than the circumference of the rod body and the circumference of the connector.

7. The break off rod of claim 1, wherein the rod body does not overhang from the spinal assembly after insertion of the break off rod.

8. The break off rod of claim 1, wherein the break off rod is inserted in the spinal assembly at a low profile.

9. The break off rod of claim 1, wherein the rod body of the break off rod is inserted in two or more screw heads of the spinal assembly.

10. The break off rod of claim 1, wherein the connector is fastened within the body of the insertion device.

11. The break off rod of claim 1, wherein the connector stays fastened to the insertion device after the connector is broken off from the rod body.

12. An insertion device for percutaneous insertion of a break off rod with a spinal assembly, the insertion device comprising:
a body, wherein the body comprises a fastener to fasten a connector of a break off rod for percutaneous insertion with a spinal assembly, wherein the break off rod comprises a rod body, a connector, and a break off region, wherein the insertion body abuts against the spinal assembly when the rod body of the break off rod is properly inserted in the spinal assembly, and wherein the insertion body is configurable to break off the connector from the rod body prior to removal of the insertion body.

13. The insertion device of claim 12, wherein the connector is cylindrical and threaded and the fastener of the insertion device is threaded and configurable to fasten the connector to the insertion device.

14. The insertion device of claim 12, wherein the connector is shaped as a flat wedge and the fastener is shaped to receive the flat wedge shaped connector and configurable to lock in the connector.

15. The insertion device of claim 12, wherein the body of the insertion device is configurable to break off the connector from the rod body at the break off region when a rotational force is applied to the insertion body.

16. The insertion device of claim 12, wherein the break off region has a smaller circumference than the circumference of the rod body and the circumference of the connector.

17. The insertion device of claim 12, wherein the rod body does not overhang from the spinal assembly after removal of the body of the insertion device.

18. The insertion device of claim 12, wherein the body of the insertion device is configurable to insert the rod body in the spinal assembly at a low profile.

19. The insertion device of claim 12, wherein the body of the insertion device is configurable to insert the rod body in two or more screw heads of the spinal assembly.

20. The insertion device of claim 12, wherein only a portion of the connector is connected within the body of the insertion device.
